(19) United States
(12) Patent Application Publication
(10) Pub. No.: US 2005/0019391 A1
(43) Pub. Date: Jan. 27, 2005

(54) Orodispersible tablet having high homogeneity and the preparation method thereof

(76) Inventors: Edouard Gendrot, Garnay (FR); Nourredine Nouri, Cannes (FR); Pascal Suplie, Montaure (FR)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(21) Appl. No.: 10/494,505

(22) PCT Filed: Oct. 28, 2002
(86) PCT No.: PCT/FR02/03690

(30) Foreign Application Priority Data
Nov. 5, 2001 (FR) 01/14269

(51) Int. Cl. A61K 9/20

(52) U.S. Cl. 424/464

(57) ABSTRACT

The invention relates to a rapid-disintegrating tablet of the type which is intended to disintegrate in the mouth on contact with the saliva in less than 40 seconds, preferably in under 30 seconds, and which forms an easy-to-swallow suspension. The inventive tablet is made from at least one active material which takes the form of coated microcrystals or microgranules and a mixture of excipients in the form of grains. The aforementioned mixture of excipients has the following composition: between 60 and 85% of a diluent; between 3 and 20% of a decomposing agent; between 1 and 8% of a sweetening agent; between 0 and 5% of a gliding agent; between 0 and 10% of a binder; and between 0 and 10% of a permeabilising agent, a swelling agent and/or a lubricant. The above-mentioned percentages are weight percentages in relation to the total weight of the grains of the excipients. Said excipient grains have a median particle size of between +30 and −30 %, and preferably between +10 and −10%, in relation to the dimension of the coated microcrystals or microgranules.

The present invention relates to an orodispersible tablet, which is a rapid release tablet which disintegrates in the mouth in less than 40 seconds or even in less than 30 seconds. The invention is also directed to the method of preparing said tablet.

Existing fast release tablets, such as those described by the company Prographarm in FR9109245, FR208642, FR9709233, and FR9814034, are satisfactory although they can be improved from the standpoint of the uniform of distribution of the active substance throughout the tablet and also from one tablet to another, particularly when the tablet's unit dose is low or moderate, representing for example less than 25% of the unit mass of the tablet. Discrepancies in the distribution of the active substance or substances throughout the tablet do arise and may induce physicochemical modifications in the tablets.

The primary aim of the invention will be to overcome these drawbacks and provide tablets having great uniform distribution of active substance throughout the tablet and also from one tablet to another while maintaining an abrasion (measured as indicated in the French pharmacopoeia (Xth edition, “V.5.1- abrasion des comprimés” [tablet abrasion], January 1993)) of less than 2% and exhibiting an excellent breakdown time in the mouth and a pleasing palatability, these being essential characteristics of the tablets described previously by the Applicant Company.

Surprisingly and unexpectedly the Applicant Company has found that all of these features can be combined in a single tablet comprising microcrystals or microgranules of coated active substance and a mixture of excipients in the form of grains by selecting an appropriate mixture of excipients and a specific particle size ratio between the microgranules and the grains of excipient.

The present invention accordingly provides rapid-release tablets which are able to disintegrate in the mouth upon contact with saliva in less than 40 seconds, preferably in less than 30 seconds, to form an easy-to-swallow suspension which are based on at least one active substance in the form of coated microcrystals or microgranules and on a mixture of excipients in the form of grains, the mixture of excipients comprising in particular:
from 60 to 85% of a diluent,
from 3% to 20% of a disintegrant,
from 1% to 8% of a sweetener,
from 0% to 5% of a flow agent,
from 0% to 10% of a binder,
from 0% to 10% of a permeabilizing agent, swelling agent and/or lubricant,
   the percentages being percentages by weight relative to the total weight of the grains of excipients,
   and the grains of excipients having a median particle size ranging between +30 and −30%, preferably between +10 and −10%, relative to the size of the coated microcrystals or microgranules.

In one advantageous embodiment of the invention the mixture of excipients may further comprise at least one flavoring agent and/or at least one colorant.

The breakdown time in the mouth corresponds to the period which elapses between the moment when the tablet is in the mouth upon contact with saliva and the moment at which the suspension resulting from the disintegration of the tablet is swallowed.

The mixture of excipients in the form of grains forming part of the tablets in accordance with the invention comprises a diluent, a disintegrant, a sweetener, a flow agent, and optionally other excipients selected from the group comprising in particular a binder, a swelling agent, a lubricant, a colorant and a flavoring agent.

The diluent forming part of the grains of excipients is selected from the group comprising in particular polyols with less than 13 carbon atoms, especially mannitol, xylitol, sorbitol, and maltitol, microcrystalline celluloses, sugars and derivatives, dicalcium phosphate and its derivatives, glycine and other pharmaceutically compatible amino acids, and their derivatives, lactose and its derivatives, and mixtures thereof.

The disintegrant is selected from the group comprising in particular crosslinked sodium carboxymethylcellulose, which is designated in the art by the term croscarmellose, crospovidone, carboxymethylstarch and mixtures thereof.

The sweetener forming part of the grains of excipients is selected from the group comprising in particular aspartame, potassium acesulfame, sodium saccharinate, neohesperidine dihydrochalcone, sucralose and mixtures thereof.

The flow agent is selected from the group comprising in particular the hydrophobic colloidal silica known under the brand name Aerosil® R 972.

The binder forming part of the grains of excipients is selected from the group comprising in particular alginic acid, sodium alginate, starch, including pregelatinized starch, carboxymethylcellulose, dextrin, gelatine, glucose syrup, guar gum, hydrogenated vegetable oils, hydroxyethylcellulose, hydroxypropylcellulose, hydroxypropylmethylcellulose, aluminum magnesium silicate, maltodextrins, methylcellulose, polyethylene oxide, polymethacrylates, povidone, copovidone, polyethylene glycols, microcrystalline celluloses, sugars and their derivatives, and mixtures thereof.

According to one advantageous embodiment of the invention the binder is selected from the group comprising alginic acid, sodium alginate, carboxymethylcellulose, hydroxypropylcellulose with a low degree of substitution, aluminum magnesium silicate, methylcellulose, starch, including pregelatinized starch, and mixtures thereof.

With particular advantage the binder is selected from the group comprising cornstarch, pregelatinized starch, hydroxypropylcellulose, maltodextrins and mixtures thereof.

The permeabilizing agent which may form part of the grains of excipients is selected from the group of silicas which have a high affinity for aqueous solvents, such as the precipitated silica available under the brand name Syloid® or the colloidal silica available under the brand name Aerosil®, maltodextrins, β-cyclodextrins, micronized polyoxyethylene glycol and its derivatives, and mixtures thereof.

According to one advantageous embodiment of the invention the permeabilizing agent is the precipitated silica known under the brand name Syloid® FP 244.

The flavoring agent and the colorant which may form part of the grains of excipients are selected from those which are pharmaceutically acceptable. They are selected in accordance with the desired organoleptic characteristics for the end product and so as best to mask the residual taste of the active substance.

The swelling agent which may be part of the grains of excipients is selected from the group comprising in particular starch, a modified starch or microcrystalline cellulose.

The lubricant which may be part of the grains of excipients is selected from the group comprising in particular stearic acid, magnesium stearate, sodium stearylfumarate, micronized polyoxyethylene glycol (micronized Macrogol 6000), leucine, sodium benzoate, and mixtures thereof.

According to another embodiment of the invention the lubricant may also be present or may be present exclusively on the surface of the final tablet.

The flavoring agent may be liquid or solid. It may be combined with taste enhancers, such as citric acid, with fresheners such as menthyl lactate and its derivatives, and with any other excipient which allows obtaining satisfactory taste qualities.

According to different methods of introduction, the flavoring agent and the colorant may be introduced either during the manufacture of the excipient granules or after the manufacture of these granules by mixing them with those granules.

According to one advantageous embodiment the flavor is introduced after the manufacture of excipient granules, by mixing it with said granules.

The tablets according to the invention are suitable for the use of any type of active substance which is in microcrystal form or can be granulated.

The active substance may be selected from the group comprising gastrointestinal sedatives, antacids, analgesics, antiinflammatories, coronary vasodilators, peripheral and cerebral vasodilators, antiinfectives, antibiotics, antivirals, antiparasitics, anticancer agents, anxiolytics, neuroleptics, central nervous system stimulants, antidepressants, antihistamines, antidiarrheal agents, laxatives, dietary supplements, immunodepressants, hypocholesterolemics, hormones, enzymes, antispasmodics, antianginal agents, medicinal products which influence heart rate, medicinal products used in the treatment of arterial hypertension, antimigraine agents, medicinal products which influence blood clotting, antiepileptics, muscle relaxants, medicinal products used in the treatment of diabetes, medicinal products used in the treatment of thyroid dysfunctions, diuretics, anorexigenic agents, anti-asthmatics, expectorants, antitussives, mucoregulators, decongestants, hypnotics, antinausea agents, hematopoietic agents, uricosuric agents, plant extracts, and contrast agents.

The tablets in accordance with the invention are particularly suitable for active substances used in treatments intended for children or for the elderly, because of the easy swallowing.

The active substance is present in the tablet in the form of coated microcrystals or microgranules.

The size of the particles of active substance varies between 10 and 30 μm.

The coating of the microcrystals or microgranules of active substance may be one of those described in patent applications FR9109245, FR9704234 and FR9806.384.

The composition of the functional coating layer is selected depending on the desired taste masking and/or active substance release characteristics.

According to one advantageous embodiment the tablets of the invention are such that the average size of the coated microcrystals or microgranules of active substance is from 100 μm to 500 μm, preferably from 200 μm to 400 μm, and the size of the grains of excipients is from 70 μm to 650 μm, preferably from 180 μm to 440 μm.

The tablets in accordance with the invention exhibit very low abrasion, less than 2% as measured according to the protocol described in the French pharmacopoeia (Xth edition, “V.5.1- abrasion des comprimés” [tablet abrasion], January 1993).

This very low abrasion makes it possible to use conventional industrial methods of transfer and of packaging of the tablets, which do not require special precautions and which can be implemented very rapidly. The packaging used, moreover, may be conventional packaging for moisture-sensitive tablets.

According to one advantageous embodiment the weight composition of the tablets in accordance with the invention is as follows:
30% to 50%, preferably 35 to 45%, of coated microcrystals or microgranules of active substance;
50% to 70%, preferably 55 to 65%, of grains of excipients;
0 to 10%, preferably less than 5%, of flavoring agent;
and 0 to 6%, preferably less than 2%, of lubricant distributed on the surface of the tablet.

The present invention likewise provides the method of preparing these tablets.

The method comprises the following steps:
preparing coated microcrystals or microgranules,
preparing grains of excipients,
optionally adding at least one flavoring agent and/or at least one colorant to the mixture of grains of excipients,
mixing the coated microcrystals or microgranules of active substance and the grains of excipients,
and tableting the mixture.

The coated microcrystals or microgranules of active substance may be prepared as described in patent applications FR9109245 and FR0014803.

The grains of excipients are prepared by wet granulation of the mixture of excipients.

According to one advantageous embodiment water is used as the wetting agent for the granulation.

The final step, that of tableting, is easy to carry out, given that the blend of coated microcrystals or microgranules and of grains of excipients is very homogeneous, and there is therefore no separation.

The tablets obtained exhibit great uniformity with one another and great uniform distribution of the excipients, and therefore exhibit very low abrasion.

Furthermore, an additional advantage is obtained by employing the present invention. Since the final tableting is very easy, it can be carried out by nonspecialized agents. It is therefore possible to convey the coated microcrystals or microgranules of active substance and the grains of excipients separately and to carry out tableting at the site of commercialization. This can be done in order to alleviate the problems of conveying products which are highly sensitive to water.

The invention may be better understood with the aid of the following examples, which are not limitative but relate to advantageous embodiments of the invention.

### EXAMPLES

### Example 1

### Preparation of Grains of Excipients

A mixer is charged with 4866 g of Mannitol 60, 850 g of Kollidon CL, 284 g of aspartame and 300 g of L-HPC LH 21. This mixture is homogenized for 5 minutes and then wetted with 2.5 l of water for 1 minute. The mixture obtained is poured into a decaking apparatus and then into a 1.5 mm screen granulator of Alexanderwerk type. The mixture is subsequently dried in an air bed dryer of Glatt GPCG3 type. The grains obtained are subsequently screened on a 0.8 mm screen.

The particle size distribution of the grains of excipients thus prepared is between 80 μm and 610 μm.

The particle size midpoint is 450 μm.

The grains are subsequently impregnated with liquid banana flavor.

### Example 2

### Preparation of Paracetamol Tablets

Microcrystals of paracetamol are introduced into a fluidized air bed apparatus and are sprayed with a dispersion of Eudragit E 100, Eudragit NE 30 D, and colloidal silica in ethanol in order to obtain coated microcrystals with 10% of polymer.

The particle size distribution of the coated microcrystals thus obtained is 290-619 μm with a peak at 440 μm.

The coated paracetamol microcrystals thus obtained are then mixed with grains of excipients as prepared in example 1, in a proportion by weight of 65% of grains of excipients to 35% of coated paracetamol microgranules.

The mixture thus obtained is tableted on a Fette-type tableting machine using 12-type punches with a compression force of 9.5 KN.

The hardness of the resulting tablets ranges from 35 to 40N. This hardness was measured by means of a breaking strength measurement apparatus of Erweka type.

The orally disintegration time is approximately 10 seconds. This time corresponds to the period which elapses between the moment when the tablet is in the mouth upon contact with saliva and the moment when the suspension resulting from the disintegration of the tablet is swallowed.

The abrasion is 0.4%. The abrasion is measured according to the protocol described in the French pharmacopoeia (Xth edition, “V.5.1- abrasion des comprimés” [tablet abrasion], January 1993).

1-10. (canceled)

11. Fast release tablet which disintegrates in the mouth upon contact with saliva in less than 40 seconds, thus resulting in an easy-to-swallow suspension which is based on at least one active substance in the form of coated microcrystals or microgranules and on a mixture of excipients in the form of grains, comprising:
from 60 to 85% of a diluent,
from 3% to 20% of a disintegrant,
from 1% to 8% of a sweetener,
from 0% to 5% of a flow agent,
from 0% to 10% of a binder,
from 0% to 10% of a permeabilizing agent, swelling agent and/or lubricant,
the percentages being percentages by weight relative to the total weight of the grains of excipients,
and in that the grains of excipients have a median particle size of between +30 and −30% relative to the size of the coated microcrystals or microgranules.

12. Tablet according to claim 11 which disintegrates in the mouth upon contact with saliva in less than 30 seconds.

13. Tablet according to claim 11, wherein the grains of excipients have a median particle size of between +10 and −10%, relative to the size of the coated microcrystals or microgranules.

14. Tablet according to claim 11, wherein the diluent is selected from the group consisting of polyols with less than 13 carbon atoms, especially mannitol, xylitol, sorbitol, and maltitol, microcrystalline celluloses, sugars and derivatives thereof, dicalcium phosphate and its derivatives, glycine and other pharmaceutically compatible amino acids, and their derivatives, lactose and its derivatives, and mixtures thereof, and in that the disintegrant is selected from the group consisting of crosslinked sodium carboxymethylcellulose, which is designated in the art by the term croscarmellose, crospovidone, carboxymethylstarch, and mixtures thereof.

15. Tablet according to claim 14, wherein the binder is selected from the group consisting of alginic acid, sodium alginate, starch, including pregelatinized starch, carboxymethylcellulose, dextrin, gelatine, glucose syrup, guar gum, hydrogenated vegetable oils, hydroxyethylcellulose, hydroxypropylcellulose, hydroxypropylmethylcellulose, aluminum magnesium silicate, maltodextrins, methylcellulose, polyethylene oxide, polymethacrylates, copovidone, povidone, polyethylene glycols, microcrystalline celluloses, sugars and their derivatives, and mixtures thereof.

16. Tablet according to claim 11, wherein the grains of excipients may further comprise a flavoring agent.

17. Tablet according to claim 16, wherein the flavoring agent is in liquid form.

18. Tablet according to claim 16, wherein the flavoring agent is in particulate form.

19. Tablet according to claim 16, wherein the flavoring agent is in pulverulent form.

20. Tablet according to claim 16, wherein the flavoring agent is added after the manufacture of the granules of excipients.

21. Tablet according to claim 11, wherein the tablet may further comprise at least one flavoring agent and/or at least one colorant.

22. Tablet according to claim 11, having a weight composition as follows:
30% to 50%, of coated microcrystals or microgranules of active substance;
50% to 70%, of grains of excipients;
0 to 10%, of flavoring agent;
and 0 to 6%, of lubricant distributed on the surface of the tablet.

23. Tablet according to claim 11, having a weight composition as follows:
35 to 45%, of coated microcrystals or microgranules of active substance;
55 to 65%, of grains of excipients;
less than 5%, of flavoring agent;
less than 2%, of lubricant distributed on the surface of the tablet.

24. Tablet according to claim 11, wherein the average size of the coated microcrystals or microgranules of active substance is from 100 μm to 500 μm, and the size of the grains of excipients is from 70 μm to 650 μm.

25. Tablet according to claim 24, wherein the average size of the coated microcrystals or microgranules of active substance is from 200 μm to 400 μm, and the size of the grains of excipients is from 180 μm to 440 μm.

26. Tablet according to claim 11, exhibiting an abrasion of less than 2%, measured as indicated in the French pharmacopoeia (Xth edition, “V.5.1- abrasion des comprimés” [tablet abrasion], January 1993).

27. A method of preparing a tablet according to claim 11, comprising the following steps:
preparing the coated microcrystals or microgranules of active substance;
wet-granulating the mixture of excipients;
optionally adding a flavoring agent and colorant to the grains of excipients;
mixing the grains of excipients thus obtained with the coated microcrystals or microgranules of active substance;
and dry-tableting the mixture thus obtained.

28. A method according to claim 27, comprising the addition of flavoring agent and colorant to the grains of excipients.
